(19) United States
(12) Patent Application Publication
(10) Pub. No.: US 2004/0204694 A1
(43) Pub. Date: Oct. 14, 2004

(54) Device and method of reducing friction and displacement of ostomy appliances

(76) Inventor: Micheal E. Nicholson, Kirkland, WA (US)

Correspondence Address: GRAYBEAL JACKSON HALEY LLP, Frederick A. Kaseburg, Suite 350, 155 - 108th Avenue NE, Bellevue, WA, 98004-5901, US

(21) Appl. No.: 10/668,939

(22) Filed: Sep. 22, 2003

Related U.S. Application Data
(60) Provisional application No. 60/462,074, filed on Apr. 10, 2003.

(51) Int. Cl. A61M 1/00

(52) U.S. Cl. 604/327

(57) ABSTRACT

An ostomy appliance cover that includes a first sheet of flexible material having at least one low coefficient of friction surface and dimensioned to cover a first portion of the ostomy appliance, and a second sheet of flexible material that is elastic and breathable, dimensioned to cover a second portion of the ostomy appliance. The second sheet of material includes an expandable opening, which when expanded allows the ostomy appliance to pass through the opening without substantial distortion of the ostomy appliance, and when released substantially covers the colostomy appliance in cooperation with the first sheet of material.

### PRIORITY

This application claims priority from U.S. provisional patent application No. 60/462,074, filed Apr. 10, 2003, which is incorporated herein by reference.

### FIELD OF THE INVENTION

The present invention relates to a cover for ostomy appliances, and in particular to a low-friction cover that reduces forces applied to the ostomy appliance by clothing and intimate contact.

### BACKGROUND

Ostomy appliances are widely used post surgical appliances for patients that have undergone a surgical procedure resulting in intestinal waste elimination through a stoma in an abdominal wall. These appliances and their use are described in U.S. Pat. No. 4,211,224, for FILTER VENTING DEVICES FOR OSTOMY APPLIANCES issued Jul. 8, 1980, and U.S. Pat. No. 5,356,399, for OSTOMY APPLIANCE issued Oct. 18, 1994, both of which are incorporated herein by reference.

The term “ostomy” covers all types of surgical procedures wherein a passageway is formed through the skin and a portion of the intestine connected thereto. When a portion of the large intestine or colon is connected to the stoma or passageway, the surgical procedure is referred to as a “colostomy”. When the small intestine is involved, the surgical procedure is known as a “ileostomy”. Both types of procedures require an ostomy appliance, such as a cap or bag which is affixed or worn on the body and is in communication with the stoma. The cap generally functions as a protective cover and limits waste material exiting from the stoma. The bag additionally provides for collection of waste products. A wide variety of ostomy caps and bags, and methods for attaching them to the body have been previously devised. For purposes of describing background and embodiments of the invention, embodiments are described with reference to an appliance used in conjunction with a colostomy.

Colostomy appliances, particularly ostomy caps (sometimes known as stoma caps), are adhered to skin adjacent to the stoma and are generally worn relatively unobtrusively beneath the wearer's clothing. Most colostomy appliances are constructed of materials that do not breath so they can perform their designed function of retaining waste products. However, the non-breathable materials used in colostomy appliances generally frictionally resist movement of the wearer's clothing over them. Because of the friction, clothing chafes, rubs, and scuffs against the appliance, sometimes resulting in folding, displacing, or disengaging the appliance from the stoma site. Any displacement or disengagement of the colostomy appliance can cause leakage, soiling, odor, and wearer embarrassment. Furthermore, the non-breathable nature of colostomy appliances causes moisture accumulation between a portion of the appliance and the wearer's skin. Additionally, the non-breathable materials can be uncomfortable against an intimate partner's skin.

In view of the foregoing, there is a need in the art for a new and improved apparatus and method for reducing friction between colostomy appliances and a wearer's clothing or an intimate partner. There is a further need for limiting moisture buildup between the colostomy appliance and the wearer's skin.

### SUMMARY

An embodiment of the present invention provides an ostomy appliance cover. The cover includes a first sheet of flexible material having at least one low coefficient of friction surface and dimensioned to cover a first portion of the ostomy appliance, and a second sheet of flexible material that is elastic and breathable, dimensioned to cover a second portion of the ostomy appliance. The second sheet of material includes an expandable opening, which when expanded allows the ostomy appliance to pass through the opening without substantial distortion of the ostomy appliance, and when released substantially covers the colostomy appliance in cooperation with the first sheet of material.

These and various other features as well as advantages of the present invention will be apparent from a reading of the following detailed description and a review of the associated drawings.

### BRIEF DESCRIPTION OF THE DRAWINGS

The features of the present invention which are believed to be novel are set forth with particularity in the appended claims. Aspects of the invention, together with further objects and advantages thereof, may best be understood by making reference to the following description taken in conjunction with the accompanying drawings, in the several figures of which like referenced numerals identify like elements, and wherein:

FIG. 1 illustrates a side view of a colostomy appliance applied to a wearer's skin in an area of a stoma;

FIG. 2 illustrates a stoma-side plan view of an ostomy appliance cover, according to an embodiment of the invention;

FIG. 3 illustrate a cross-section side view of an ostomy appliance cover, according to an embodiment of the invention; and

FIG. 4 illustrates a side view of the colostomy appliance of FIG. 1 with a colostomy appliance cover fitted over the colostomy appliance, according to an embodiment of the invention.

### DETAILED DESCRIPTION

In the following detailed description of exemplary embodiments of the invention, reference is made to the accompanying drawings, which form a part hereof. The detailed description and the drawings illustrate specific exemplary embodiments by which the invention may be practiced. These embodiments are described in sufficient detail to enable those skilled in the art to practice the invention. It is understood that other embodiments may be utilized, and other changes may be made, without departing from the spirit or scope of the present invention. The following detailed description is therefore not to be taken in a limiting sense, and the scope of the present invention is defined by the appended claims.

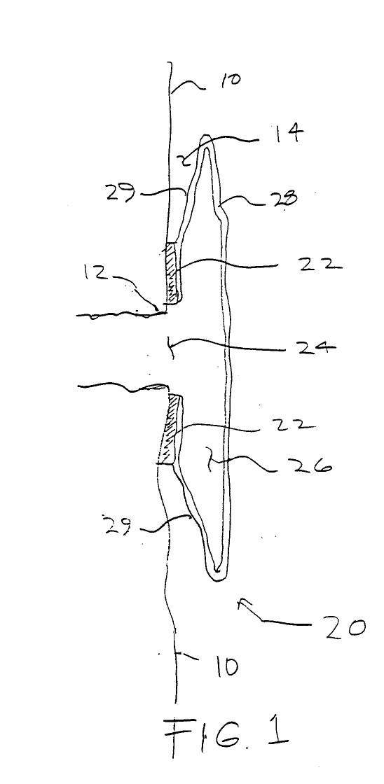

FIG. 1 is a side view of a colostomy appliance 20, illustrated by a cap, applied to a wearer's skin 10 in an area of a stoma 12 of the human body. The colostomy appliance 26 may be round, rectangular, or any other shape known in the art suitable for performing the function of the colostomy appliance. The colostomy appliance 20 may be any type of appliance for fitting over the stoma 12, such as the COLOPLAST® 5540, 5560 & 5700, and the CONVATEC® 22710. The appliance 20 includes an outer-surface portion 28 and a wearer's skin-side portion 29.

The skin-side portion 29 includes an adhesive portion 22 that surrounds an inlet 24 of the appliance 20, and that is arranged for removable adhesive attachment to the wearer's skin 10 and for mating alignment with the wearer's stoma 12. The appliance 20 further includes a chamber 26 for receiving gases and waste products discharged from the stoma 12. The outer surface 28 of the colostomy appliance faces away from the wearer's skin 10 and will contact the wearer's clothing or a person in intimate contact with the wearer. The colostomy appliance 20, including the outer surface portion 28 and the skin-side portion 29, is typically made from non-breathable materials that frictionally resist movement of the wearer's clothing relative to the appliance. Furthermore, perspiration and moisture typically form in area 14 between the wearer's skin 10 and the skin-side portion 29 of the colostomy appliance 26.

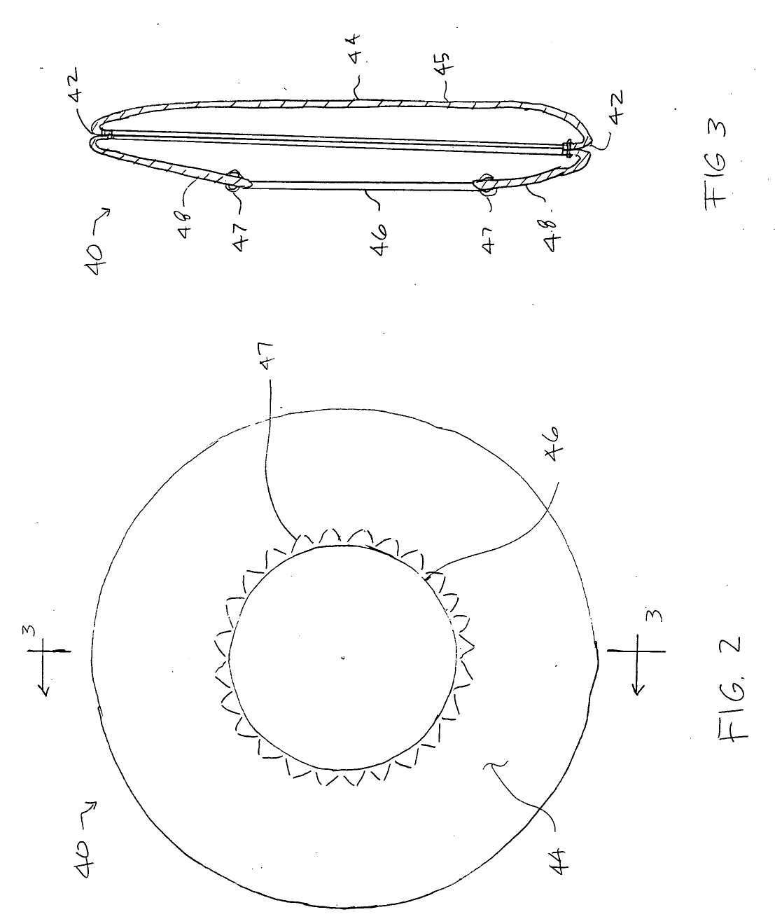

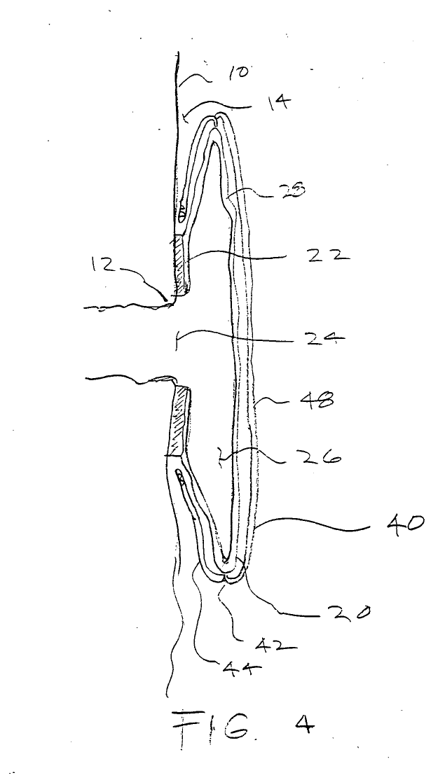

FIGS. 2 and 3 illustrate a stoma-side plan view and a cross-section side view respectively of an ostomy appliance cover 40, according to an embodiment of the invention. The cover 40 includes a sheet of material 44 having a low coefficient of friction surface 45, and a sheet of elastic, breathable material 48. The sheet of breathable material 48 includes an expandable opening 46 having a seam 47 around a peripheral portion of the opening providing reinforcement of the opening and additional elasticity.

The sheet of material 44 having the low coefficient of friction surface 45 is configured for a close fit with the outer surface 28 of the appliance 20. The low coefficient of friction surface 45 is orientated facing away from the outer surface 28 and for contact with the wearer's clothes and persons in intimate contact with the wearer. The sheet of elastic, breathable material 48 is configured for a close fit with the skin-side surface of the cover 40.

The sheet of low coefficient of friction material 44 and the sheet of elastic, breathable material 48 each have an outer peripheral edge, and are coupled together at their respective outer peripheral edges by the seam 42 to cooperatively form the cover 40. The seam 42 can be formed in manner known in the art, including stitching, gluing, or both. In an alternative embodiment, the cover 40 may be made from a single piece of material arranged to cover the colostomy appliance 20.

The cover 40, including sheets 44 and 48, is optimally configured for a close fit with the appliance 20 to reduce movement of the cover with respect to the appliance 20 while still allowing for expansion of the appliance for gas and waste products. Additionally, sheets 44 and 48 are optimally configured to lay proximate to the outer surface 28 and the skin-side surface 29 of the appliance 20 without wrinkles.

The colostomy appliance cover 40 may be constructed of any materials suitable for its intended purpose known to those in the art, particularly materials that are lightweight, longwearing, and washable. The flexible material 44 having a low coefficient of friction surface 45 may be any flexible sheet or planar material with at least one surface having a low coefficient of friction with respect to clothing or human skin. The low coefficient of friction surface 45 may be a surface of a material having a smooth, silky or satiny surface. Such a surface may include low-friction fibers, a combination of fibers resulting in a low-friction surface, or a material modified to have a low-friction surface. The flexible material may be a film, sheet, or woven material, and may be natural, synthetic, organic, or inorganic. A low coefficient of friction surface 45 generally has a coefficient of friction of less than 0.3. The flexible material 44 may also be breathable.

The sheet of elastic, breathable material 48 may be any elastic, breathable material known to those in the art, including fabric materials with a weave that allows breathability, and materials perforated for breathability. The material 48 includes sufficient elasticity for the expandable opening 46 to be pulled over an outside diameter (not shown) of a colostomy appliance 10 without significantly distorting the appliance. The edge of the opening 46 can be surged with a flexible stitching to prevent unraveling while allowing the colostomy appliance cover 40 to be easily put on and removed. The colostomy appliance cover 40 may be constructed in a variety of colors and utilize a variety of decorative patterns.

An example of an ostomy appliance cover was made to cover a round Coloplast model 5540 stoma cap cover having a 4.6-inch overall outside diameter, and an outside diameter around the adhesive portion 22 of 2.8 inches. A commonly available Lycra Spandex® material having at least one low coefficient of friction surface 45 was used for both the flexible material 44 and the sheet of elastic, breathable material 48. The two sides are cut such that their outer peripheries, when sewn together by seam 42, form an inner diameter (not shown) of approximately 4.8 inches. Generally, it has been found that the cover 40 functions well when between one-sixteenth and one-fourth-inch larger than the stoma appliance cover 20, with one-eighth-inch being a preferred dimension. The opening 46 in the material 48 is cut for a finished diameter of approximately 2.4 inches; being slightly smaller than the 2.8-inch outside diameter of the adhesive portion 22 and providing a small, elastic gripping of the appliance cover 20 by the stoma cap cover 40 at the adhesive portion. The opening 46 is reinforced with stitching 47 in a zigzag pattern about the opening with an elastic thread at about 8-12 stitches per inch. The seam 42 is sewn in a straight pattern with an elastic thread at 8-12 stitches per inch. Typically, the two pieces of material are sewn together inside out, and then turned right side out to provide a finished edge and look.

FIG. 4 is side view of the colostomy appliance 20 of FIG. 1 with the colostomy appliance cover 40 of FIGS. 2 and 3 fitted over the colostomy appliance 20, according to an embodiment of the invention.

The cover 40 may be applied to the appliance 20 either before or after the appliance 20 is adhered to a patient's skin 10. For example, after the appliance 20 is adhered to the patient's skin 20 by the adhesive portion 22, the expandable opening 46 is expanded by stretching and flattening with the wearer's fingers to a point where the opening 46 is substantially flat and approximately equal to, or larger than, an outside dimension of the appliance 20. By way of further example using the embodiment of the invention constructed as described in conjunction with FIGS. 2 and 3 with the 4.6-inch diameter round appliance, the expandable opening 46 is expanded into a slit configuration having a length longer than 4.6 inches. The cover 40 is then pulled over the appliance 20 in a motion from the patient's feet toward the patient's head. Because of the elasticity in at least the material 48 comprising the opening 46, the cover 40 with the expanded opening configuration easily fits over the appliance 20 without substantial distortion of the worn appliance. When released from the expanded opening configuration, the cover 40 substantially covers the cap 20.

Once the cover is applied, the elastic, breathable material 48 allows moisture to move away and helps to prevent friction, chafing, and irritation of skin 10 area around the stoma 12. The low coefficient of friction surface 45 prevents the appliance 20 from folding or wadding up under the wearer's clothing, and keeps the appliance 20 lying close to the skin. One of the many advantage to this feature is that “wadding and folding” can lead to premature failure of the cap-sealing features. Additionally, it is much easier to wear close-fitting clothing without embarrassing bulges. In addition, the colostomy appliance cover also provides some protection of clothes and keeps an undergarment from soiling when minor leaks and failures occasionally occur. Furthermore, the colostomy appliance cover 40 promotes more comfortable intimate contact by helping to maintain adhesion to the wearer's skin, eliminating potential binding against a partner's skin, and providing a more attractive appearance than the non-breathable material from which an ostomy cap is typically manufactured.

Additional advantages of the colostomy appliance cover 40 include being reusable and easy to maintain. It launders easily due to the material it is constructed from, such as lycra spandex which dries quickly for reuse. A spare colostomy appliance cover 40 can be carried unobtrusively on one's person.

Although the present invention has been described in considerable detail with reference to certain preferred embodiments, other embodiments are possible. Therefore, the spirit or scope of the appended claims should not be limited to the description of the embodiments contained herein. It is intended that the invention resides in the claims hereinafter appended.

What is claimed is:

1. An ostomy appliance cover, comprising:
a first sheet of flexible material having at least one low coefficient of friction surface and dimensioned to cover a first portion of the ostomy appliance; and
a second sheet of flexible material that is elastic and breathable, dimensioned to cover a second portion of the ostomy appliance, and includes an expandable opening, which when expanded allows the ostomy appliance to pass through the opening without substantial distortion of the ostomy appliance, and when released substantially covers the colostomy appliance in cooperation with the first sheet of material.

2. The cover of claim 1, wherein the first and second sheets of material each have an outer peripheral edge, and the sheets of material are coupled together at their respective outer peripheral edges.

3. The cover of claim 1, wherein the first sheet material further includes a configuration to lay proximate to the first portion of the appliance without substantial wrinkling.

4. The cover of claim 1, wherein the second sheet of material further includes a configuration to lay proximate to the second portion of the appliance without substantial wrinkling.

5. The cover of claim 1, wherein the opening includes a configuration that exposes an ostomy inlet of the ostomy appliance.

6. The cover of claim 1, wherein the ostomy appliance includes an ostomy cap.

7. The cover of claim 1, wherein a periphery of the expandable opening includes a stretchable stitching.

8. The cover of claim 1, wherein a periphery of the expandable opening includes a ring of an elastic material.

9. The cover of claim 1, wherein in the low coefficient of friction surface has a coefficient of friction of less than 0.3.

10. The cover of claim 1, wherein the second sheet of material includes a woven fabric.

11. The cover of claim 1, wherein the first sheet of material includes a woven fabric.

12. The cover of claim 1, wherein the first and second sheets of material comprise a same material.

13. The cover of claim 1, wherein the opening includes a configuration that when expanded allows the ostomy appliance when worn by a user to pass through the opening without substantial distortion.

14. An ostomy appliance cover operable to:
reduce friction between a first portion of the ostomy appliance and a surface other than a skin surface of a wearer of the ostomy appliance;
separate a second portion of the ostomy appliance from the skin surface of the wearer and reduce moisture formation between the skin surface and the ostomy appliance; and
fit over the ostomy appliance without dislodging the ostomy appliance from the skin of the user while the ostomy appliance is worn by the user.

15. A method of reducing a displacement tendency of a ostomy appliance worn by a person, the method comprising steps of:
placing on an ostomy appliance a material that reduces friction between a first portion of the worn ostomy appliance and clothing of the person; and
placing on an ostomy appliance a material that reduces moisture formation between skin of the person and the second portion of the appliance.
